(19) United States
(12) Patent Application Publication
(10) Pub. No.: US 2024/0428923 A1
(43) Pub. Date: Dec. 26, 2024

(54) SYSTEMS AND METHODS FOR MULTI-PATIENT VENTILATION LIBERATION

(71) Applicant: KONINKLIJKE PHILIPS N.V., EINDHOVEN (NL)

(72) Inventors: MARTIJN SPIT, DELFT (NL); JACCO CHRISTOF EERDEN, VLEUTEN (NL); FEI ZUO, EINDHOVEN (NL); ESTHER MARJAN VAN DER HEIDE, 's-Hertogenbosch (NL); THOMAS MARIA FALCK, AACHEN (DE)

(21) Appl. No.: 18/749,841

(22) Filed: Jun. 21, 2024

Related U.S. Application Data
(60) Provisional application No. 63/509,354, filed on Jun. 21, 2023.

(51) Int. Cl. G16H 20/40; A61M 16/00; G16H 10/40; G16H 10/60; G16H 40/20; G16H 50/30

(52) U.S. Cl. CPC G16H 20/40; A61M 16/0051; A61M 16/0066; A61M 16/026; G16H 10/40; G16H 10/60; G16H 40/20; G16H 50/30; A61M 2205/10; A61M 2205/3561; A61M 2205/505; A61M 2230/04; A61M 2230/208; A61M 2230/42

(57) ABSTRACT

A method for ventilation liberation is provided, including: (1) generating readiness statuses for each of a plurality of patients; (2) generating a risk score for each of the plurality of patients; and (3) generating patient planning data, such as patient assignments for staff members and/or a suggested treatment schedule. The readiness statuses may include SAT, SBT and/or extubation readiness statuses. The readiness statuses are based on patient characteristics such as vital sign measurements, ventilator settings, laboratory data, primary diagnosis data, sedation status, and/or patient medication data. The risk score is based on patient risk factors such as vital sign measurements, primary diagnosis data, patient comorbidity data, and/or reintubation risk data. The patient assignments and/or the suggested treatment schedule are based on the readiness statuses of each of the patients, the risk score of each of the patients, and/or staffing data corresponding to each of the staff members.

### FIELD OF THE DISCLOSURE

The present disclosure is generally directed to operational intelligence solutions in the intensive patient care domain and, more particularly, to systems and methods for multi-patient ventilation liberation.

### BACKGROUND

Ventilation liberation (also referred to as weaning from mechanical ventilation) is the process of reducing patient sedation and ventilatory support, ultimately resulting in the patient breathing spontaneously and being extubated. Important steps in the ventilation liberation process are Spontaneous Awakening Trials (SAT) and Spontaneous Breathing Trials (SBT). The decision to start an SAT and/or an SBT is currently based on safety criteria that includes multiple factors, such as oxygenation level of the ventilator, pressure levels of the ventilator, use of vasopressors, the presence of myocardial ischemia, how awake the patient is, etc. In addition, intensive care facility staff must be available to initiate the trials, monitor the patient during the trials, and perform extubation if the patient is ready. Failure to initiate ventilation liberation in a timely manner can lead to an increased duration of mechanical ventilation and may result in complications such as ventilator associated pneumonia (VAP) or other adverse events such as self-extubation, all of which may extend the length of stay of the patient.

Performing SATs and SBTs, coupled with improved and more frequent screenings for SAT and SBT eligibility will help reduce time to ventilation liberation. However, several issues regarding SAT and SBT often arise. First, in healthcare environments, where SATs and SBTs are not carried out, ventilation liberation is often done too conservatively. In these environments, patients may be ready to spontaneously breathe and be extubated, but this is delayed because staff members erroneously believe they are not ready. Second, in healthcare environments where SAT and SBT are being used, such as intensive care units, patients may not be screened for SAT and/or SBT eligibility on a regular basis (such as daily). The lack of regular eligibility screening may be due to the staff members being busy with other tasks, management not prioritizing SAT and/or SBT, staff finding it difficult to include SAT and/or SBT eligibility screening in their workflow, too many patients being eligible for SBT at the same time, etc. Third, even if SAT and SBT eligibility screening is performed daily, the screening is usually performed once per day at a fixed point in time. However, patients may be ready for SAT and/or SBT at any time of day. Failure to quickly identify eligibility may unnecessarily extend mechanical ventilation. Fourth, staff members are not always aware as to the extent that their interventions affect weaning readiness. For example, patients may be given sedation at night to reduce agitation, but this sedation may also delay SAT and/or SBT eligibility for one or more days.

### SUMMARY OF THE DISCLOSURE

The present disclosure is generally directed to operational intelligence solutions in the intensive patient care domain and, more particularly, to systems and methods for multi-patient ventilation liberation. The systems and methods use a variety of information related to patients receiving respiratory support (“ventilator patient”) and staff members of a healthcare facility to determine readiness of the patients for ventilation interventions, risk scores associated with the patients, and patient planning data. The patient planning data can include patient assignments for the staff members to perform the interventions and/or a suggested treatment schedule. The systems and methods use these determinations to generate a variety of virtual dashboards informing the staff members of the status of the patients and the optimized assignments and schedules for the ventilation interventions.

A patient overview module determines readiness statuses for each ventilator patient. The readiness statuses indicate whether the patient is ready for spontaneous awakening trials (SATs), spontaneous breathing trials (SBTs), and/or extubation (collectively referred to herein as “ventilation intervention”). The readiness statuses are determined based on patient characteristics derived from patient monitoring devices, electronic medical records (EMRs), and/or ventilators. The patient characteristics may include vital sign measurements, ventilator settings, laboratory data, primary diagnosis data, sedation status, and/or patient medication data for each patient in the healthcare facility currently on a ventilator. Accordingly, these readiness statuses may be continually updated throughout the patient's term on the ventilator without requiring staff member intervention.

The patient overview module also determines risk scores for each ventilator patient. The risk scores are used to prioritize ventilation interventions for patients who are more at risk than others. The risk scores are based on patient risk factors derived from the patient monitoring devices and the EMRs corresponding to the ventilator patients. The patient risk factors may include vital sign measurements, primary diagnosis data, patient comorbidity data, and/or reintubation risk data. As with the readiness statuses, the risk scores may be continually updated throughout the patient's term on the ventilator without requiring staff member intervention.

Further, a staff overview module receives information regarding each staff member of the healthcare facility which may be involved in ventilation interventions. The staff overview module synthesizes this information into staffing data. This staffing data is used to both assign individual patients to individual staff members and schedule a time for the intervention (whether it be SAT, SBT, or extubation). The staffing data may include work schedule, workload, patient assignments, staff role, and/or staff experience level for each staff member which may be tasked to perform ventilation interventions.

A planning module then uses the information generated from the patient overview module and the staff overview module to generate patient planning data. In one example, the patient planning data includes patient assignments for at least a portion of the staff members. The patient assignments indicate the pairing of the staff member to the patient. The patient assignments are generated based on the readiness statuses of the patients, the risk scores of the patients, and the staffing data. For example, a patient with a higher risk score may be assigned to a more experienced staff member.

Further, the patient planning data may also include suggested treatment schedules for the ventilator patients based on the patient assignments. The suggested treatment schedule may include a prioritized action order for the interventions, optimized timing information for each intervention, and/or schedule adjustment recommendations to ensure each patient is assigned to the proper staff member. The planning module may also generate staff alerts based on the suggested treatment schedule. For example, a staff alert may be generated when a patient assigned to the staff member is ready for a ventilation intervention. The staff alert may be sent to a mobile device operated by the staff member, to a bedside patient monitor, or to a centralized staff member station.

The information generated by the patient overview module, the staff overview module, and the planning module may be used by a dashboard module to generate visual dashboards to be shown on a user interface. In one example, the dashboard module generates an individual patient readiness dashboard displaying readiness statuses and patient characteristics for a single patient. Alternatively, the dashboard module may also generate a multi-patient readiness dashboard displaying readiness statuses and patient characteristics for two or more patients. Further, the dashboard module may also generate a suggested schedule dashboard displaying information regarding the suggested treatment schedule.

Generally, in one aspect, a method for ventilation liberation is provided. The method includes generating, via a controller having a processor and a memory, one or more readiness statuses for each of a plurality of patients. The one or more readiness statuses are generated based on a plurality of patient characteristics. According to an example, the one or more readiness statuses may include an SAT readiness status, an SBT readiness status, and/or an extubation readiness status. According to another example, the plurality of patient characteristics may include vital sign measurements, ventilator settings, laboratory data, primary diagnosis data, sedation status, and/or patient medication data.

The method further includes generating, via the controller, a risk score for each of the plurality of patients. The risk score is generated based on a plurality of patient risk factors. According to an example, the plurality of patient risk factors may include vital sign measurements, primary diagnosis data, patient comorbidity data, and/or reintubation risk data.

The method further includes generating, via the controller, patient planning data corresponding to the plurality of patients. The patient planning data is based on the one or more readiness statuses of each of the plurality of patients, the risk score of each of the plurality of patients, and/or staffing data corresponding to a plurality of staff members. The staffing data may include work schedule, workload, patient assignments, staff role, and/or staff experience level. The patient planning data may include one or more patient assignments for one or more staff members of the plurality of staff members. The patient planning data may also include a suggested treatment schedule.

According to an example, the method further includes displaying, via a user interface, an individual patient readiness dashboard. The individual patient readiness dashboard may display at least one of the plurality of patient characteristics and at least one of the one or more readiness statuses of one of the plurality of patients.

According to an example, the method further includes displaying, via a user interface, a multi-patient readiness dashboard. The multi-patient readiness dashboard displays at least one of the one or more readiness statuses of two or more of the plurality of patients.

According to an example, the method further comprises generating, via the controller, a suggested schedule dashboard displaying the suggested treatment schedule.

According to an example, the method, further comprises generating, via, the controller, one or more staff alerts based on the suggested treatment schedule.

According to an example, the method further comprises generating, via the controller, one or more readiness interventions for one or more of the plurality of patients based on the plurality of patient characteristics.

Generally, in another aspect, a ventilation liberation system is provided. The ventilation liberation system includes a controller. The controller has a processor and a memory. The controller is configured to generate one or more readiness statuses for each of a plurality of patients based on a plurality of patient characteristics.

The controller is further configured to generate a risk score for each of the plurality of patients based on a plurality of patient risk factors.

The controller is further configured to generate patient planning data corresponding to the plurality of patients. The patient planning data is based on the one or more readiness statuses of each of the plurality of patients, the risk score of each of the plurality of patients, and/or staffing data corresponding to a plurality of staff members.

In various implementations, a processor or controller may be associated with one or more storage media (generically referred to herein as “memory,” e.g., volatile and non-volatile computer memory such as RAM, PROM, EPROM, EEPROM, floppy disks, compact disks, optical disks, magnetic tape, SSD, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a functional block diagram of a system for ventilation liberation, in accordance with an example.

FIG. 2 is another functional block diagram of a system for ventilation liberation evaluation, in accordance with an example.

FIG. 3 is a table showing readiness scores, risk scores, patient characteristics, and patient assignments associated with two patients, in accordance with an example.

FIG. 4 is a user interface displaying an individual patient readiness dashboard, in accordance with an example.

FIG. 5 is a user interface displaying a multi-patient readiness dashboard, in accordance with an example.

FIG. 6 is a user interface displaying a suggested schedule dashboard, in accordance with an example.

FIG. 7 is a schematic diagram of a controller of a system for ventilation liberation, in accordance with an example.

FIG. 8 is a schematic diagram of aspects of a memory of the controller of FIG. 7, in accordance with an example.

FIG. 9 is a flow chart of a method for ventilation liberation, in accordance with an example.

### DETAILED DESCRIPTION OF EMBODIMENTS

The present disclosure is generally directed to operational intelligence solutions in the intensive patient care domain and, more particularly, to systems and methods for multi-patient ventilation liberation. The systems and methods use a variety of information related to patients receiving respiratory support (“ventilator patient”) and staff members of a healthcare facility to determine readiness of the patients for ventilation interventions, risk scores associated with the patients, and patient planning data. The patient planning data can include patient assignments for the staff members to perform the interventions and/or a suggested treatment schedule. The systems and methods use these determinations to generate a variety of virtual dashboards informing the staff members of the status of the patients and the optimized assignments and schedules for the ventilation interventions.

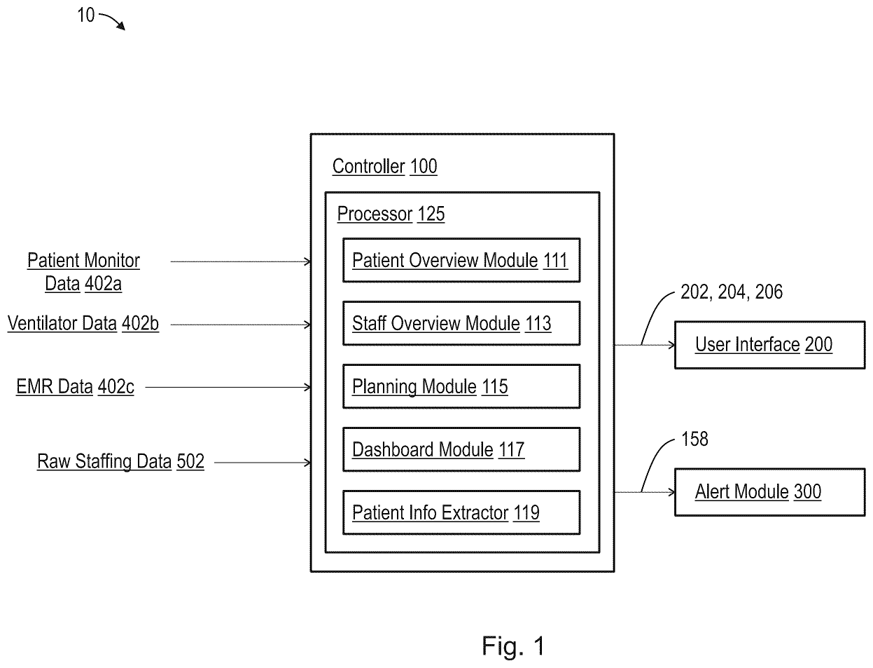

FIG. 1 illustrates a system 10 for multi-patient ventilation liberation, according to various embodiments of the invention. Generally, the system 10 evaluates data collected regarding one or more patients P to determine if the liberation process may begin, as well as to determine if any particular patients P should be prioritized due to certain risk factors. The system 10 also collects information regarding the staff members SM of the healthcare facility, and assigns some of the staff members SM to the patients P to perform the liberation processes. The system 10 may then generates a variety of dashboards regarding individual patients P, multiple patients P, and intervention schedules to be displayed on a user interface. Accordingly, the system 10 is enabled to continuously determine if the patients P are ready for ventilation liberation, and if so, to assign staff members SM to perform the liberation interventions based on risk factors associated with the patients P.

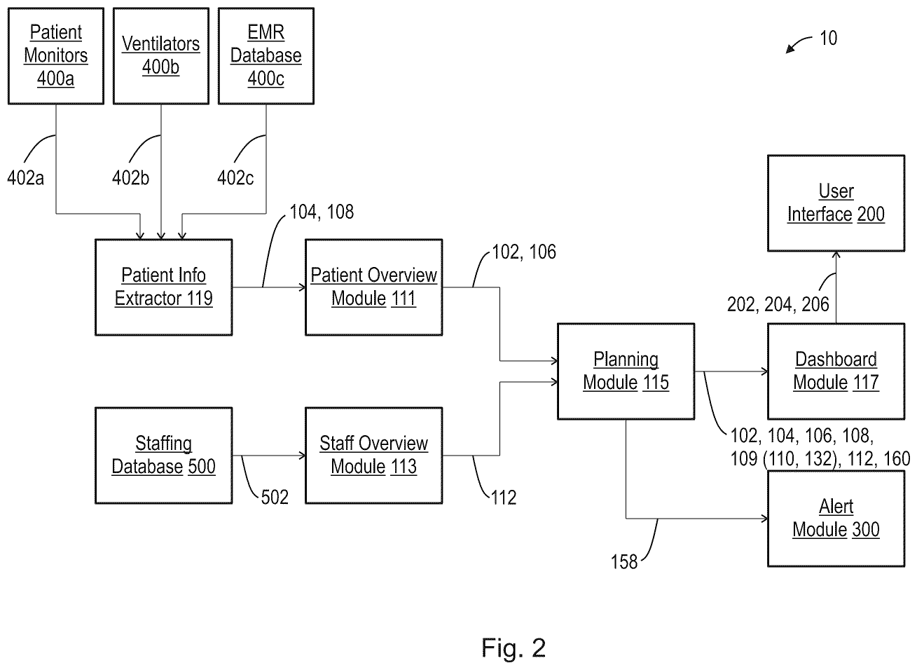

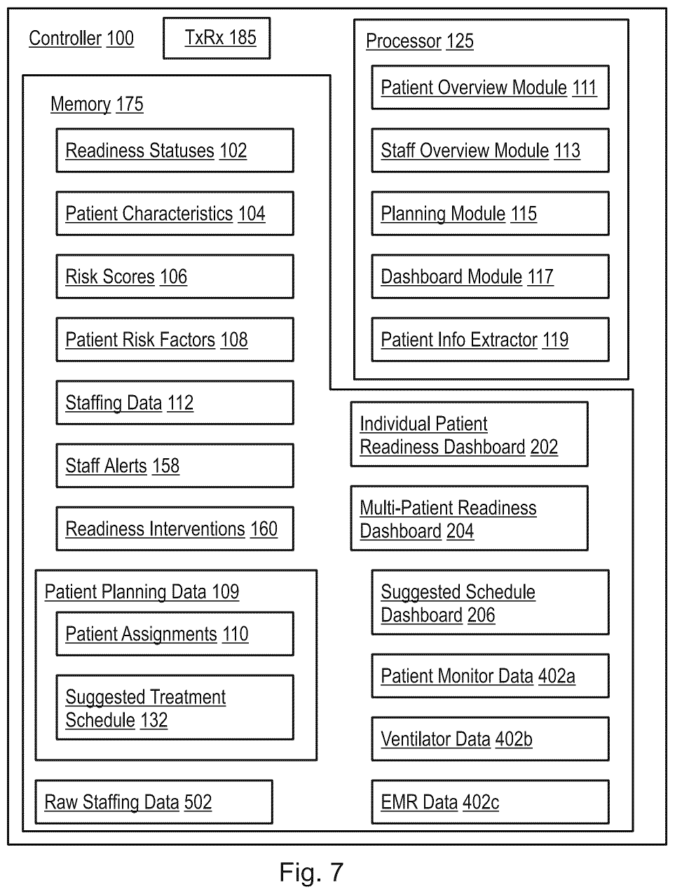

With continued reference to FIG. 1, the system 10 includes a controller 100. The controller 100, shown in more detail in FIG. 7, includes a processor 125, a memory 175, and a transceiver 185. The processor 125 is configured to execute a variety of modules to estimate or predict various aspects of the system 10. In the example of FIG. 1, the processor 125 is configured to execute a patient overview module 111, a staff overview module 113, a planning module 115, a dashboard module 117, and a patient information extractor 119. These modules 111, 113, 115, 117, 119 are described in further detail with reference to FIG. 2. The memory 175 is configured to store data received by the controller 100 via wired or wireless connection. The memory 175 is further configured to store data generated by the processor 125.

The transceiver 185 is configured to wirelessly transmit data to or wirelessly receive data from various aspects of the system 10. The transceiver 185 may also enable wireless communication with components arranged externally to the system 10. As shown in more detail in the example of FIG. 2, the transceiver 185 may wirelessly receive a variety of data, including patient monitor data 402a, ventilator data 402b, electronic medical record (EMR) data 402c, and raw staffing data 502. As will be explained in more detail with respect to FIG. 2, the system 10 extracts and processes the aforementioned information to determine if a plurality of patients P is ready for ventilation liberation interventions, to determine the level of risk associated with the interventions when performed on a particular patient, and to determine the optimum patient assignments and schedules for the staff members SM.

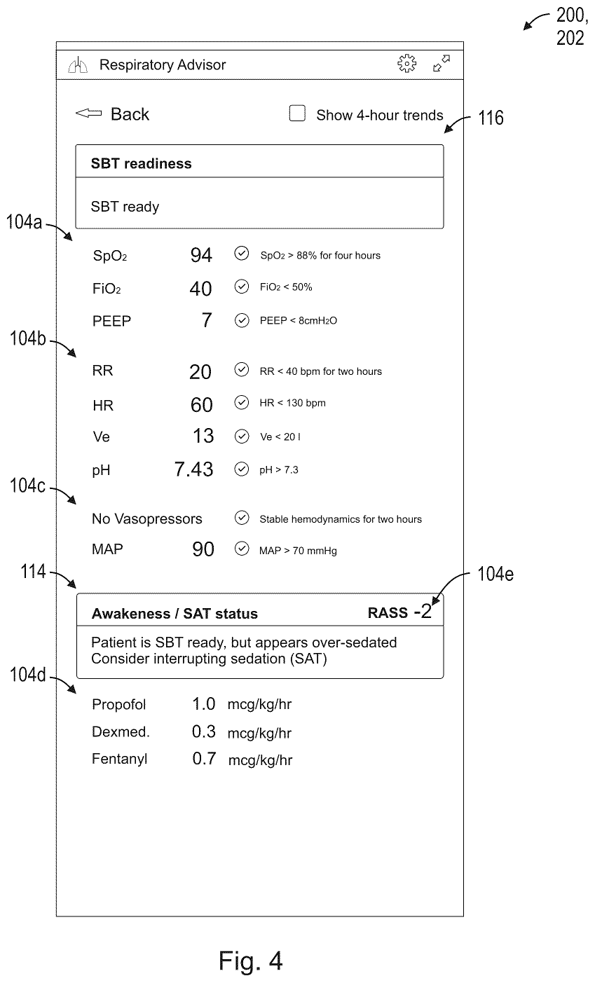

The system 10 also includes a user interface 200. Non-limiting examples of the user interface 200 are shown in FIGS. 4-6. In some examples, the user interface 200 may include a display screen to show various dashboards 202, 204, 206 generated by the controller 100 and displaying various information related to the patients P and staff members SM. The dashboards 114 may be conveyed to the user interface 200 via wired or wireless connection, such as via the transceiver 185 of the controller 100. The user interface 200 may be any practical type of display, such as a touch screen display. In some examples, the user interface 200 may be a component of the controller 100.

The system 10 also includes an alert module 300. The alert module 300 may be configured to convey staff alerts 158 regarding ventilator liberation interventions to mobile devices (such as smartphones or tablet computers) operated by the staff members SM. In some examples, the alert module 300 may be a component of the controller 100.

FIG. 2 is another functional block diagram of a system 10 for ventilation liberation. In particular, FIG. 2 illustrates the flow of data between the various models and programs executed by the processor 125 of the controller 100 to generate recommendations if and when a patient P should be liberated from a ventilator, as well as which staff member SM should perform the liberation, according to various embodiments of the invention.

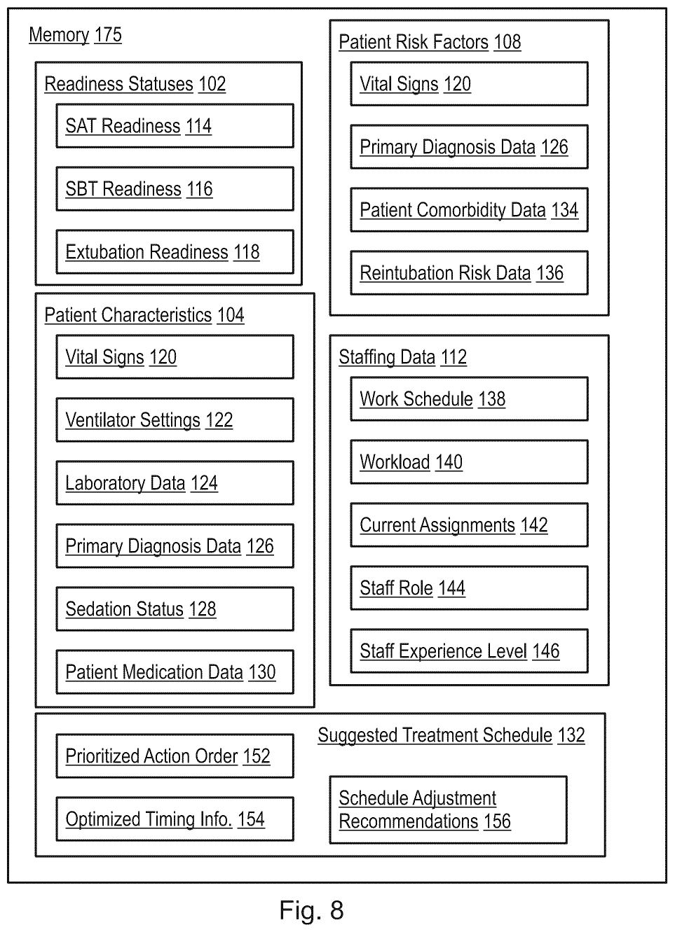

As shown in FIG. 2, the system 10 includes a patient overview module 111. The patient overview module 111 is configured to determine one or more readiness statuses 102 of a patient P, as well as a risk score 106 associated with the patient P. The patient P has been intubated, meaning that an endotracheal tube has been inserted into the trachea of the patient P. The tube is connected to a mechanical ventilator to deliver air or oxygen, thereby assisting the breathing of the patient P. As shown in FIG. 8, the readiness statuses 102 may include spontaneous awakening trial (SAT) readiness status 114, spontaneous breathing trial (SBT) 116, and/or extubation readiness status 118. SAT and SBT are important steps in determining if a patient P is ready for extubation. In SAT, sedating medications used to treat an intubated patient P are temporarily paused to determine if the patient P requires continual sedation or if the patient P can be managed without sedatives. In SBT, mechanical ventilation is paused to determine if the intubated patient P may be able to breathe without ventilator assistance (or with minimal assistance). Extubation is the last step of ventilation liberation, where the endotracheal tube is removed from the patient P.

SAT readiness status 114 and SBT readiness status 116 are determined based on a variety of patient characteristics 104. These patient characteristics 104 may include information such as vital sign measurements 120, ventilator settings 122, laboratory data 124, primary diagnosis data 126, sedation status 128, and/or patient medication data 130. As seen in FIG. 2, the patient characteristics 104 are derived from information automatically and/or continuously captured from a variety of sources. First, bed-side patient monitors 400a continuously capture patient monitor data 402a regarding the patient P while they are intubated, such as information regarding vital sign measurements 120. Second, the ventilator 400b used to intubate the patient P continuously records ventilator data 402b corresponding to the patient P, such as information regarding ventilator settings 122. Third, an electronic medical record (EMR) database 400c continuously updates EMR data 402c regarding the patient P collected throughout their stay, such as laboratory data 124, primary diagnosis data 126, sedation status 128, and/or patient medication data 130. Accordingly, the data collected by the patient monitor 400a, the ventilator 400b, and the EMR database 400c enables the system 10 to automatically determine if a patient qualifies for SAT and/or SBT without continual surveillance by a staff member SM.

The patient monitor data 402a, the ventilator data 402b, and the EMR data 402c are provided to the controller 100 via a wired or wireless connection. The patient information extractor 119 of the controller 100 then converts the patient monitor data 402a, the ventilator data 402b, and the EMR data 402c into patient characteristics 104 capable of being processed by the patient overview module 111.

Vital sign measurements 120 are typically captured by the patient monitor 400a using sensors placed on or proximate to the patient P. Vital sign measurements 120 may include information regarding oxygen saturation, blood pressure, heart rate, respiration rate, electrocardiogram data, and more. For example, vital sign measurements 120 corresponding to patient instability may indicate that the patient P is not ready for spontaneous awakening or breathing trials. The measured blood pressure may be a mean arterial pressure (MAP).

The ventilator settings 122 reflect the mechanical settings used by the ventilator 400b to assist the patient P in breathing. In some examples, the ventilator settings 122 are manually programmed by a staff member SM. In other examples, the ventilator settings 122 may automatically adjust during the assisted breathing process. The ventilator settings 122 may include information regarding fraction of inspire oxygen (FiO₂), positive end-expiratory pressure (PEEP), minute ventilation (Ve), and more.

Laboratory data 124 may include a wide array of testing data corresponding to the patient P, such as blood tests. The blood may be obtained by an arterial blood gas draw via an arterial line of an IV system or a syringe. The blood test may include information regarding pH level, carbon dioxide level, oxygen level, bicarbonate level, and/or oxygen saturation level, as well as the presence or level of any other substance in the blood of the patient P. The laboratory data 124 may be stored in the EMR data 402c corresponding to the patient P.

Primary diagnosis data 126 corresponds to the disorder, disease, injury, or ailment experienced by the patient P leading to the intubation. For example, the patient P may have experienced respiratory failure, cardiac arrest, airway obstruction, apnea, etc. The primary diagnosis data 126 may be stored in the EMR data 402c corresponding to the patient P.

Sedation status 128 may provide information regarding the sedation level of the patient P. In some cases, if a patient P is too heavily sedated, they may be unable to awaken during SAT. The sedation status 128 may be tracked in EMR data 402c, and, in some cases, may be derived from data captured by the patient monitor 402a.

Patient medication data 130 provides information regarding medication the patient P is currently taking or has previously taken. For example, during intubation, the patient P may be taking some combination of vasopressors, propofol, dexmedetomidine, and/or fentanyl. The patient medication data 130 may be tracked in EMR data 402c, and, in some cases, may be derived from laboratory data 124.

Having received the aforementioned patient characteristics 104 from the patient information extractor 119, the patient overview module 111 determines the readiness statuses 102 for the patient P. For example, the patient P may have an SAT readiness status 114 indicating that the patient P is not currently eligible for SAT, as they are too heavily sedated to awaken. In other examples, the patient P may have an SBT readiness status 116 indicating that the patient P is eligible for SBT due to stable vital sign measurements 120 and ventilation settings 122.

Extubation readiness status 118 is determined based on the results of the SAT and SBT trials, as well as aspects of the other patient characteristics 104. For example, if the patient P successfully completed SAT and SBT, the extubation readiness status 118 may indicate that the patient P is eligible for extubation. However, despite completing SAT and SBT, the patient may have other characteristics, such as suddenly unstable vital sign measurements 120 indicating that they may not be eligible for extubation.

In addition to the readiness statuses 102, the patient overview module 111 also determines a risk score 106 for each patient P. While a patient P may be eligible for SAT, SBT, or extubation, the patient P may have certain traits increasing the risk associated with the intervention. This risk is indicated by the risk score 106. The patient overview module 111 determines the risk score 106 based on patient risk factors 108 provided by the patient information extractor 119. Like the patient characteristics 104, the patient risk factors 108 are also derived from information captured and/or stored by the patient monitors 400a, the ventilators 400b, and/or the EMR database 400c. The risk score 106 is used by the system 10 to prioritize ventilation intervention (SAT, SBT, or extubation) of patients P associated with higher risk. Further, staff members 112 having higher skill levels and/or more experience may be assigned to perform the interventions on patients P having higher risk scores 106.

The patient risk factors 108 may include vital sign measurements 120, primary diagnosis data 126, patient comorbidity data 134, and reintubation risk data 136. The vital sign measurements 120 and the primary diagnosis data 126 are described above in regard to determining the readiness statuses 102. The patient comorbidity data 134 corresponds to patient characteristics, diseases, or disorders which can increase patient risk during SAT, SBT, or extubation. The patient comorbidity data 134 may be derived from EMR data 402c. The reintubation risk 136 quantifies the risk that the patient P may need to be intubated again following extubation, meaning that the patient P is not yet capable of breathing on their own. The reintubation risk 136 may be based on a wide array of different patient risk factors, including vital sign measurements 120 (such as respiration rate) and patient comorbidity data 134 (such as age).

The planning module 115 uses the readiness status 102 and risk score 106 associated with each patient P to generate patient planning data 109 corresponding to the patient P. In some examples, the patient planning data 109 includes patient assignments 110 for staff members SM of the healthcare facility. The patient assignments 110 may be generated based on, in part, staffing data 112. The staffing data 112 is provided to the planning module 115 via a staffing overview module 113. The staffing overview module 113 processes raw staffing data 502 from a staffing database 500 to be used by the planning module 115. The staffing database 500 may be communicatively coupled to the controller 100 via wired and/or wireless connection. The staffing data 112 may include a wide array of information associated with the staff members SM, such as work schedule 138, workload 140, current assignments 142, staff role 144, and staff experience level 146. The work schedule 138 is descriptive of the upcoming tasks the staff member SM must complete over a period of time in the future. The workload 140 indicates a volume of upcoming tasks and/or patients P. Current assignments 142 provide more detail regarding patients P already assigned to be treated by the staff member SM. Staff role 144 indicates the title of the staff member SM within the care facility, such as doctor, nurse, specialist, technician, etc. Experience level 146 indicates the amount of experience of the staff member SM. The experience level 146 may account for experience in their current position and/or experience in one or more former positions.

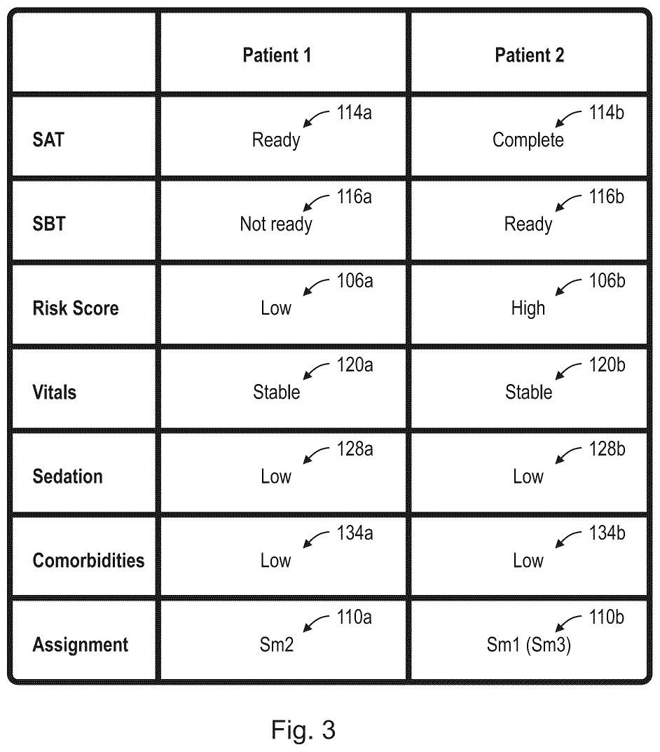

In one illustrative example shown in the table of FIG. 3, a first SAT readiness status 114a indicates that a first patient P1 is ready for SAT due to, at least in part to, stable vital sign measurements 120a and a low-level sedation status 128a. Further, a first risk score 106a indicates the first patient P1 as low risk due to their patient comorbidity data 134a indicating a lack of significant comorbidities. Further, a second SBT readiness status 116b indicates a second patient P2 is ready for SBT. However, a second risk score 106b indicates the second patient P2 as high risk do to, in part, advanced age and a history of respiratory issues.

The staffing data 112 (as shown in FIGS. 2 and 8) indicates that three staff members SMs, a first nurse SM1, a second nurse SM2, and a doctor SM3, are available according to their associated work schedules 138 (as shown in FIG. 8). While both nurses SM1, SM2 are qualified to perform SAT and SBT, the first nurse SM1 has significantly more experience than the second nurse SM2. Accordingly, due to the high-risk associated with the second patient P2, the first nurse SM1 is assigned to perform SBT on the second patient P2, while the second nurse SM2 is assigned to perform SAT on the first patient P1. If the first nurse SM1 is unable to perform the intervention due to other factors (such as a heavy workload 140), the doctor SM3 may be assigned to replace the first nurse SM1 and perform the SBT on the second patient P2.

In some examples, the patient planning data 109 may also include a suggested treatment schedule 132. The suggested treatment schedule 132 may be particularly important when sufficient staff members SM are not immediately available to perform interventions on all patients P ready for SAT, SBT, or extubation. The suggested treatment schedule 132 may include information such as prioritized action order 152, optimized timing information 154, and schedule adjustment recommendations 156. The prioritized action order 152 prioritizes the interventions (SAT, SBT, extubation) based, at least in part, on the risk score 106 associated with the patient P. For example, SBT on a high-risk patient P_{HR} may be prioritized over SAT on a low-risk patient P_{LR}. The optimized timing information 154 may indicate a preferred day and/or time-of-day to perform an intervention. The schedule adjustment recommendations 156 are generated to suggest potential alterations of the work schedules 138 of one or more staff members SM to accommodate one or more interventions. For example, a schedule adjustment recommendation 156 may be made regarding an occupied experienced nurse if a high-risk patient P is ready for an intervention. This schedule adjustment may move a task currently assigned to the experienced nurse SM1 to another nurse SM2, allowing the experienced nurse SM1 to perform the intervention on the high-risk patient P.

In some examples, a suggested treatment schedule 132 may be generated by the planning module 115 solely based on the readiness statuses 102 and the risk scores 106 for patients P currently intubated. This feature may be particularly useful when certain types of the staffing data 112, such as the work schedules 138 of some staff members SM is unavailable. In this case, the planning module 115 may create a schedule for SAT and/or SBT based on the readiness statuses 102 of each patient P, prioritized based on the risk score 106 associated with each patient P. For example, a first patient P1 and a second patient P2 may be scheduled for SAT at 5 AM, a third patient P3 may be scheduled for SBT at 8 AM, and then the first patient P1 and the second patient P2 may be scheduled for SBT at 9 PM.

In some examples, the planning module 115 may generate staff alerts 158 based on the patient assignments 112. For example, if a first staff member SM1 is assigned to perform an SAT intervention on a second patient P2, the planning module 115 may generate a staff alert 158 to be displayed on a mobile device (such as a smartphone or tablet computer) of the first staff member SM1. The staff alert 158 may contain a wide variety of information, such as identifying information of the second patient P2, identifying information of the first staff member SM1, and the type of intervention (SAT, SBT, extubation) to be performed. The alert module 300 may wirelessly transmit the staff alert 158 to the relevant staff member SM. In other examples, the alert module 300 may provide the staff alert 158 to a bedside patient monitor or to a central monitoring location.

In some examples, the planning module 115 may be further configured to generate one or more readiness interventions 160. The readiness interventions 160 are actions to be taken by the staff members SM to ready otherwise unready patients P for interventions. For example, if a patient P is too highly sedated to initiate SAT, the readiness intervention 160 may, if doing so is safe, reduce the level of sedation such that the patient P may awaken during SAT.

The planning module 115 conveys a wide variety of data to a dashboard module 117. The dashboard module 117 is configured to convert this data into visual displays regarding patient status, patient assignments to staff, and scheduled interventions. As shown in FIG. 2, the dashboard module 117 may receive readiness statuses 102, patient characteristics 104, risk scores 106, patient risk factors 108, patient planning data 109 (including patient assignments 110 and/or suggested treatment schedules 132) staffing data 112, and suggested patient interventions 160. The dashboard module 117 uses this information to generate a variety of dashboards, including an individual patient readiness dashboard 202, a multi-patient readiness dashboard 204, and a suggested schedule dashboard 206. Non-limiting examples of these dashboards 202, 204, 206 are described in more detail with reference to FIGS. 4-6.

The system 10 further includes a user interface 200. The user interface 200 may be a display screen configured to show the aforementioned dashboards 202, 204, 206. In some examples, the user interface 200 may be embedded into a control center or control station of the hospital or healthcare facility. For example, the user interface 200 may receive, via wired or wireless transmission, and subsequently display, the dashboards 202, 204, 206 from the controller 100.

FIG. 4 illustrates an example of the user interface 200 of FIG. 2. The user interface 200 of FIG. 4 may be a display screen, such as a touch screen. The user interface 200 may be embedded in an existing control station or console, or it may be a part of a portable device, such as a tablet computer or mobile device. The example user interface 200 of FIG. 4 displays a non-limiting example of an individual patient readiness dashboard 202. The individual patient readiness dashboard 202 displays an SAT readiness status 114 and an SBT readiness status 116. These statuses 114, 116 are generated based on, at least in part, the various patient characteristics 104 displayed in the individual patient readiness dashboard 202.

The patient characteristics 104 shown in FIG. 4 are divided into five sections. The first section of patient characteristics 104a includes measured oxygen saturation (SpO₂), fraction of inspired oxygen (FiO₂) supplied to the patient P, and positive end-expiratory pressure (PEEP) of the ventilator 400b. The second section of patient characteristics 104b includes measured respiration rate (RR), measured heart rate (HR), minute ventilation (Ve), and PH level. The third section of patient characteristics 104c includes vasopressors count and mean arterial pressure (MAP). The checkmarks next to each patient characteristic 104 of the first three sections 104a, 104b, 104c indicates a value or measurement sufficient for SBT. Accordingly, the SBT readiness status 116 is shown as “SBT ready.”

The fourth section of patient characteristics 104d indicates the current levels of propofol, dexmedetomidine, and fentanyl currently being administered to the patient P. The fifth section of patient characteristics 104e indicates the Richmond Agitation Sedation Scale (RASS) of the patient P. Based on the fourth and fifth sections 102d, 102e, the patient P is not ready for SAT. Accordingly, the individual patient readiness dashboard 202 displays “Patient is SBT ready, but appears over-sedated” and “Consider interrupting sedation.”

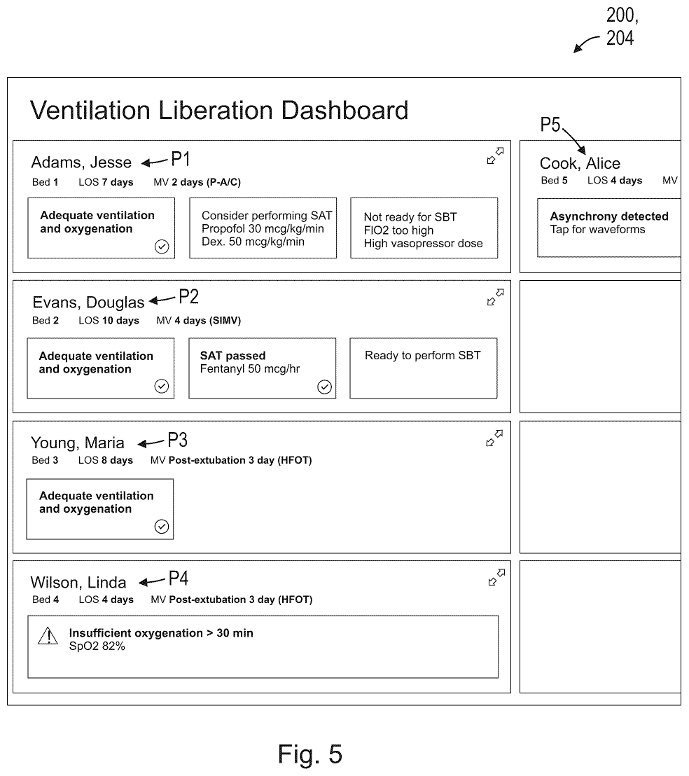

FIG. 5 illustrates a further example of the user interface 200 showing a multi-patient readiness dashboard 204. The non-limiting example of the multi-patient readiness dashboard 204 of FIG. 5 displays information regarding five patients, Jesse Adams (first patient P1), Douglas Evans (second patient P2), Maria Young (third patient P3), Linda Wilson (fourth patient P4), and Alice Cook (fifth patient P5). For each patient P, the multi-patient readiness dashboard 204 displays a bed number, a length of stay (LoS), and a mechanical ventilation period. Further, up to three windows are displayed for each patient regarding SAT readiness status 114 and SBT readiness status 116.

For example, the first patient P1 has occupied bed 1 for a length of stay of 7 days. The first patient has been under mechanical ventilation for two days, currently in pressure assist/control (P-A/C) mode. A first window informs the user that the first patient P1 is experiencing adequate ventilation and oxygenation. A second window informs the user that the first patient P1 is ready for SAT, and provides the current dosage of propofol and dexmedetomidine supplied to the first patient P1. A third window informs the user that the first patient P1 is not ready for SBT, as the FiO₂ level of the first patient P1 is too high, and that the first patient P1 is receiving a high dosage of vasopressor. In sum, the first patient P1 has a positive SAT readiness status 114, but a negative SBT readiness status 116.

The second patient P2 has occupied bed 2 for a length of stay of 10 days, and has been under mechanical ventilation for four days, currently in synchronized intermittent mandatory ventilation (SIMV) mode. A first window informs the user that the second patient P2 is experiencing adequate ventilation and oxygenation. A second window informs the user that the second patient P2 has passed SAT. A third window informs the user that the second patient P2 is now ready for SBT.

The third patient P3 has occupied bed 3 for a length of stay of 8 days, and has been extubated for three days while undergoing high-flow oxygen therapy (HFOT). A first window informs the user that the third patient P3 is experiencing adequate ventilation and oxygenation. As the third patient P3 has already been extubated, SAT and SBT statuses are inapplicable.

The fourth patient P4 has occupied bed 4 for a length of stay of 4 days, and, like the third patient P3, has also been extubated for three days while undergoing HFOT. However, the multi-patient readiness dashboard 204 indicates a warning that the fourth patient P4 is insufficiently oxygenated, thus urging staff members SM to take corrective action.

The fifth patient P5 has occupied bed 5 for a length of stay of 5 days. The multi-patient readiness dashboard 204 indicates an issue with the data collection (“Asynchrony detected”), thus urging staff members SM to take corrective action.

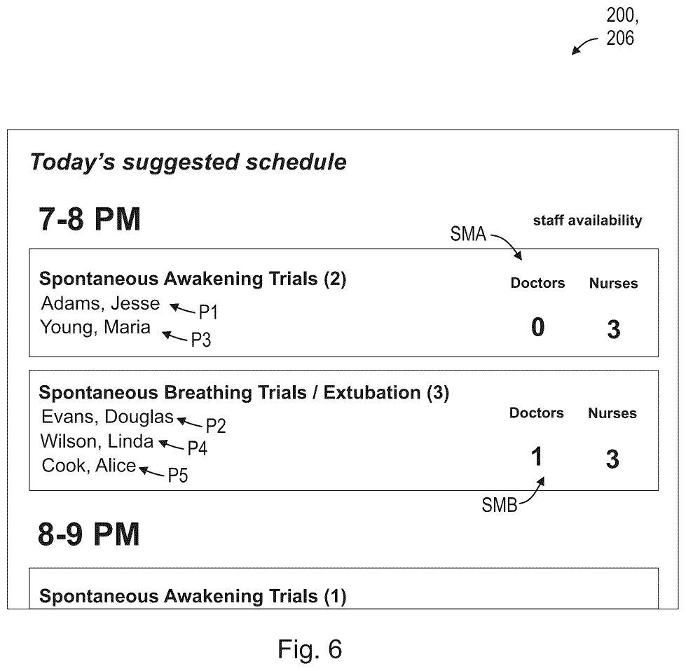

FIG. 6 illustrates a further example of the user interface 200 showing a suggested treatment schedule dashboard 206. The non-limiting example of the suggested treatment schedule dashboard 206 shows a first and a third patient P1, P3 as ready for SAT, and a second, a fourth, and a fifth patient P2, P4, P5 as ready for SBT and extubation during the 7:00 μm to 8:00 pm time window. A first group of three staff members SMA are available to perform SAT, in the form of three nurses. A second group of four staff members SMB are available to perform SBT, in the form of one doctor and three nurses. The staff members SMA, SMB may be assigned to the patients P1, P2, P3, P4, P5 according to risk score 106, with the riskiest patients P1, P2, P3, P4, P5 assigned to the most experienced and/or most proficient of the staff members SM1, SM2.

FIG. 7 schematically illustrates the controller 100 previously depicted in FIG. 1. The controller 100 includes the processor 125, the memory 175, and the transceiver 185. The memory 175 is configured to store the readiness statuses 102, the patient characteristics 104, the risk scores 106, the patient risk factors 108, the patient planning data 109 (including the patient assignments 110 and the suggested treatment schedule 132), the staffing data 112, the staff alerts 158, the readiness interventions 160, the individual patient readiness dashboards 202, the multi-patient readiness dashboards 204, the suggested schedule dashboards 206, the patient monitor data 402a, the ventilator data 402b, the EMR data 402c, and the raw staffing data 502. The processor 125 is configured to execute the patient overview module 111, the staff overview module 113, the planning module 115, the dashboard module 117, and the patient information extractor 119.

FIG. 8 schematically illustrates further aspects of the memory 175 of FIG. 7. The readiness statuses 102 include SAT readiness status 114, SBT readiness status 116, and extubation readiness status 118. The patient characteristics 104 include vital sign measurements 120, ventilator settings 122, laboratory data 124, primary diagnosis data 126, sedation status 128, and patient medication data 130. The patient risk factors 108 include vital sign measurements 120, primary diagnosis data 126, patient comorbidity data 134, and reintubation risk data 136. The staffing data 112 includes work schedule 138, workload 140, current assignments 142, staff role 144, and staff experience level 146. The suggested treatment schedule 132 includes prioritized action order 152, optimized timing information 154, and schedule adjustment recommendations 156.

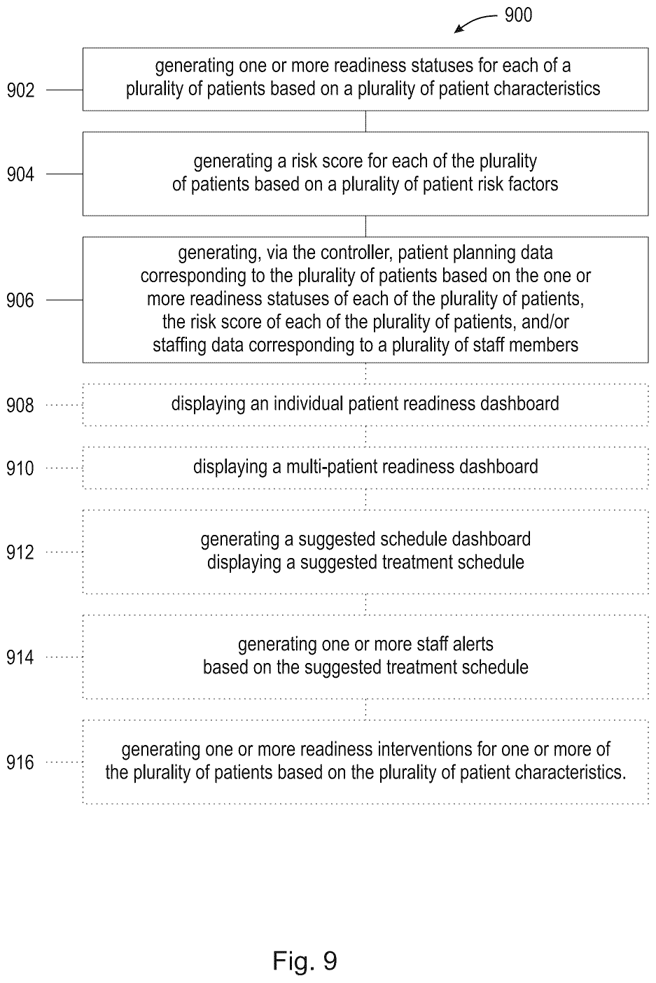

FIG. 9 is a flow chart of a method 900 for ventilation liberation of one or more patients in a healthcare facility, according to various embodiments of the invention. Referring to FIGS. 1-9, the method 900 includes, in step 902, generating one or more readiness statuses 102 for each of a plurality of patients P based on a plurality of patient characteristics 104.

The method 900 further includes, in step 904, generating a risk score 106 for each of the plurality of patients P based on a plurality of patient risk factors 108.

The method 900 further includes, in step 906, generating patient planning data 109 corresponding to the plurality of patients P based on the one or more readiness statuses 102 of each of the plurality of patients P, the risk score 106 of each of the plurality of patients P, and/or staffing data 112 corresponding to a plurality of staff members SM. The patient planning data 109 may include one or more patient assignments 110 for one or more staff members SM of the plurality of staff members SM. The patient planning data 109 may also include a suggested treatment schedule 132.

According to an example, the method 900 further includes, in optional step 908, displaying an individual patient readiness dashboard 202.

According to an example, the method 900 further includes, in optional step 910, displaying a multi-patient readiness dashboard 204.

According to an example, the method 900 further includes, in optional step 912, generating a suggested schedule dashboard 206 displaying the suggested treatment schedule 132.

According to an example, the method 900 further includes, in optional step 914, generating one or more staff alerts 158 based on the suggested treatment schedule 132.

According to an example, the method 900 further includes, in optional step 916, generating one or more readiness interventions 160 for one or more of the plurality of patients P based on the plurality of patient characteristics 104.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects may be implemented using hardware, software, or a combination thereof. When any aspect is implemented at least in part in software, the software code can be executed on any suitable processor or collection of processors, whether provided in a single device or computer or distributed among multiple devices/computers.

The present disclosure may be implemented as a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant may be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples may be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A method for ventilation liberation, comprising:
generating, via a controller having a processor and a memory, one or more readiness statuses for each of a plurality of patients (P) based on a plurality of patient characteristics;
generating, via the controller, a risk score for each of the plurality of patients (P) based on a plurality of patient risk factors; and
generating, via the controller, patient planning data corresponding to the plurality of patients (P) based on the one or more readiness statuses of each of the plurality of patients (P), the risk score of each of the plurality of patients (P), and/or staffing data corresponding to a plurality of staff members (SM).

2. The method of claim 1, wherein the one or more readiness statuses comprises a spontaneous awakening trial (SAT) readiness status, a spontaneous breathing trial (SBT) readiness status, and/or an extubation readiness status.

3. The method of claim 1, wherein the plurality of patient characteristics comprises vital sign measurements, ventilator settings, laboratory data, primary diagnosis data, sedation status, and/or patient medication data.

4. The method of claim 1, wherein the plurality of patient risk factors comprises vital sign measurements, primary diagnosis data, patient comorbidity data, and/or reintubation risk data.

5. The method of claim 1, wherein the staffing data includes work schedule, workload, patient assignments, staff role, and/or staff experience level.

6. The method of claim 1, further comprising displaying, via a user interface, an individual patient readiness dashboard.

7. The method of claim 6, wherein the individual patient readiness dashboard displays at least one of the plurality of patient characteristics and at least one of the one or more readiness statuses of one of the plurality of patients (P).

8. The method of claim 1, further comprising displaying, via a user interface, a multi-patient readiness dashboard.

9. The method of claim 8, wherein the multi-patient readiness dashboard displays at least one of the one or more readiness statuses of two or more of the plurality of patients (P).

10. The method of claim 1, wherein the patient planning data includes one or more patient assignments for one or more of the plurality of staff members (SM).

11. The method of claim 1, wherein the patient planning data includes suggested treatment schedule.

12. The method of claim 11, further comprising generating, via the controller, a suggested schedule dashboard displaying the suggested treatment schedule.

13. The method of claim 11, further comprising generating, via, the controller, one or more staff alerts based on the suggested treatment schedule.

14. The method of claim 1, further comprising generating, via the controller, one or more readiness interventions for one or more of the plurality of patients (P) based on the plurality of patient characteristics.

15. A ventilation liberation system comprising a controller having a processor and a memory, wherein the controller is configured to:
generate one or more readiness statuses for each of a plurality of patients (P) based on a plurality of patient characteristics;
generate a risk score for each of the plurality of patients (P) based on a plurality of patient risk factors; and
generate patient planning data corresponding to the plurality of patients (P) based on the one or more readiness statuses of each of the plurality of patients (P), the risk score of each of the plurality of patients (P), and/or staffing data corresponding to a plurality of staff members (SM).
